(19) United States
(12) Patent Application Publication
(10) Pub. No.: US 2025/0134362 A1
(43) Pub. Date: May 1, 2025

(54) ENDOSCOPE STEERING WIRE TENSIONING METHOD

(71) Applicant: AMBU A/S, Ballerup (DK)

(72) Inventor: Martin Johst CHRISTENSEN, Copenhagen 0 (DK)

(73) Assignee: AMBU A/S, Ballerup (DK)

(21) Appl. No.: 18/924,624

(22) Filed: Oct. 23, 2024

(30) Foreign Application Priority Data
Oct. 26, 2023 (DE) 10 2023 129 549.7

(51) Int. Cl. A61B 1/005

(52) U.S. Cl. CPC A61B 1/0057; A61B 1/0053

(57) ABSTRACT

A method for tensioning endoscope steering wires includes providing a semi-assembled endoscope including a handle and an insertion cord having a distal bending section connected to a first steering wire and a second steering wire, each steering wire having a first wire end being connected at the bending section at a distal end thereof and arranged in a respective wire pipe extending from a proximal end of the bending section to the handle, each steering wire having a proximal end extending further out of the wire pipe, providing a wire tensioner comprising a first and second wire gripper, providing a wire fastener, securing the semi-assembled endoscope relative to the wire tensioner and wire fastener, arranging each wire gripper to grip a respective one of the steering wires, activating the wire tensioner to tension the steering wires, and activating the wire fastener to fixate the steering wires in the tensioned state.

### CROSS-REFERENCE TO RELATED APPLICATIONS

This application claims priority from and the benefit of German Patent Application No. 10 2023 129 549.7, filed Oct. 26, 2023; the disclosure of said application is incorporated by reference herein in its entirety.

### TECHNICAL FIELD

The present disclosure relates to manufacturing of endoscopes and, in particular, to an endoscope steering wire tensioning method and a wire tensioning machine.

### BACKGROUND

Endoscopes are known and used for visual navigation into, and examination and diagnosis of, hollow organs and body cavities, as well as, optionally, to assist in surgery, e.g. for targeted tissue sampling. Endoscopes include procedure-specialized endoscopes, such as gastroscopes and bronchoscopes. Endoscopes may comprise a handle at the proximal end to be gripped by an operator and a flexible, elongated insertion cord extending distally from the handle. The insertion cord may include an insertion tube, a highly bendable bending section controllable by an operator, and a distal tip extending distally from the bending section. The distal tip may comprise an observation optical system. Alternatively, the endoscope may comprise an interface at the proximal end for cooperation with drive means, e.g. for implementation of robotic endoscopy.

The bending section may be controllable by steering wires connected to the bending section at the distal end and extending to the handle at the proximal end. An actuator or control mechanism, such as a wheel or lever, at the handle allows the operator to tension or slack the steering wires, thereby bending the bending section. The steering wires may be arranged inside wire pipes or sheaths. When the ends of the wire pipe are held stationary, movement of the proximal end of the steering wire with respect to the wire pipe is transmitted to the distal end as a corresponding movement of the distal end of the steering wire with respect to the wire pipe. A number of steering wires may be provided to bend the bending section in a certain direction, such as in two opposite directions or in four directions, e.g. two opposite directions in a first plane and two opposite directions in a second plane, potentially at right angles to the first plane.

Thus, using the actuator allows the operator to position the distal tip of the endoscope at a desired location by bending the bending section, advancing the elongated insertion cord, and twisting the elongated insertion cord.

Proper bending performance and feel of the endoscope are important quality parameters of an endoscope for the operator. Preferably, bending should provide direct transfer of forces from the actuator to the bending section, without rotation at the actuator that does not result in bending of the bending section, i.e. without actuator play. Thus, the steering wires should be pre-tensioned to remove slack but, on the other hand, not pre-tensioned excessively, which could strain the construction, increase friction, and deteriorate the bending performance and feel. Cost is an important parameter generally and of particular importance in single-use endoscopes, which are designed to be discarded after performing a procedure in a patient rather than being cleaned and re-used. Because assembly labor is a driver of cost, pre-tensioning should be performed in a cost-effective manner.

### SUMMARY

On this background it is an object of the disclosure to address problems such as cost, operator safety, and steering wire tension quality, during the manufacture of endoscopes, particularly single-use endoscopes, and even more particularly dual-plane single-use endoscopes (also referred to as 4-way bending endoscopes).

provide a method suitable for automation of at least some aspects of wire tensioning, to

In a first aspect, the object is achieved by a method of making the endoscope. The endoscope produced by the method should satisfy high performance standards while reducing production costs, particularly in single-use endoscopes, to increase their value. In a second aspect, the object is achieved by an endoscope manufactured by the method according to the first aspect. In a third aspect, the object is achieved by a machine and a system suitable to implement the method according to the first aspect.

Advantages of the method, machine and system include a faster manufacturing process, separation of endoscope securement from steering wire tensioning, such that an operator can secure one endoscope while the machine tensions the steering wires of another, and variation reductions in the steering wire tensions, which increases the quality of the endoscopes, for example by reducing performance variations between them.

In one embodiment according to the first aspect, the method comprises securing a subassembly of an endoscope to an endoscope pallet, mounting the endoscope pallet to a wire tensioning machine, and causing the wire tensioning machine to perform a process including tensioning a steering wire of the subassembly, sensing tension in the steering wire, and responsive to the tension exceeding a tension threshold, crimping ends of the steering wire.

The subassembly may comprise a handle and an insertion cord having a bending section. The steering wire may comprise a first steering wire and a second steering wire, the first steering wire and the second steering wire extending from a distal end of the insertion cord to the handle and being secured to the distal end such that tensioning, during use, of the first steering wire and/or the second steering wire causes the bending section to bend. The subassembly may comprise a steering actuator comprising a roller mounted on a half-shell of a housing of the handle.

The wire tensioning machine may comprise a wire gripper and a wire fastener. The wire gripper tensions the steering wire and the wire fastener fastens the end of the steering wire to itself or to the roller or the steering actuator. In one example, the wire fastener crimps the end of the steering wire to itself forming a loop secured to the roller of the steering actuator.

In a variation of the first embodiment, the method comprising: providing a semi-assembled endoscope comprising a handle and an insertion cord extending in a distal direction from the handle, the insertion cord having a distal bending section connected to a first steering wire and a second steering wire, each steering wire having a first wire end being connected at the bending section at a distal end thereof and arranged in a respective wire pipe extending from a proximal end of the bending section to the handle, each steering wire having a proximal end extending further out of the wire pipe; providing a wire tensioner comprising a first and second wire gripper; providing a wire fastener; securing the semi-assembled endoscope relative to the wire tensioner and wire fastener; arranging each wire gripper to grip a respective one of the steering wires; activating the wire tensioner to tension the steering wires; and activating the wire fastener to fixate the steering wires in the tensioned state.

Although the wires are referenced to as first and second wire, the first and second wires may comprise one wire, which at or near the middle of the length thereof forms a bend that is fixed at the distal end of the endoscope and forms two wire ends extending in the direction of the proximal end of the endoscope. The first wire extends from the bend to the first wire end, and the second wire extends from the bend to the second wire end. When assembling the endoscope, care needs to be taken that the steering wires have the correct length from the steering actuator to the bending section and the correct tension so that there is no slack. That is to say, the length of the steering wires should preferably ensure that a neutral middle position of the steering actuator corresponds to a straight, unbent, state of the bending section.

Tensioning of the wires may take place sequentially or, in one example, at least some tensioning of the wires takes place concurrently. Concurrent tensioning may be beneficial in reducing a potential risk of the two wires having different tension, which could lead to the insertion cord having a non-straight configuration in the neutral position or different bending performance in the two directions. It is presently preferred that the tensioning of the two wires takes place within +/−10 seconds of each other.

Fixation of the steering wires may take place by any suitable method, such as heat staking, gluing, soldering, by means of a fixing screw etc. In a variation of the present embodiment, fixation of the steering wires is performed by crimping, which is considered a very quick and robust type of fixation. Crimping should be understood as arranging a crimp shell over wire portions of the wire and permanently deforming the crimp shell to fixedly secure the wire portions to each other by squeezing the wire portions within the crimp shell. In this way, two overlapping wire portions may be fixated to each other, thereby ruling out any translation between the wire portions. In addition, an adhesive may be added to the wire portions inside the crimp shell to further secure the wire portions in the crimp shell to each other. The steering wires may be made of any suitable material, such as metal, e.g. steel. The steering wires may be comprised of polymers and may comprise a single polymer strand or multiple strands each. As an example, the diameter of the steering wire may be between 0.30 mm and 0.15 mm, depending on the strength of the material, preferably between and including 0.20 mm to 0.25 mm. Because the diameter of the bending section can be very small, in the range of 2.0-3.0 mm, space for the steering wires is very limited and smaller steering wire diameters are preferred.

The semi-assembled endoscope may be mounted directly in a wire tensioning machine. However, to further facilitate automation, the foregoing embodiment of the method may further comprise: providing a separate endoscope fixture; securing the semi-assembled endoscope to the fixture; and matching the semi-assembled endoscope on the fixture to the wire tensioner and wire fastener.

Providing a separate fixture could increase speed in production, as securing the semi-assembled endoscope would be independent of the wire tensioner and wire fastener, and the potentially manual process of securing the semi-assembled endoscope would not require a stop of the machinery but could be made off-line, and potentially the securing step could be performed to two or more endoscopes in individual fixtures in parallel and fed to the machinery.

According to a variation of the foregoing embodiments and variations according to the first aspect, the method comprises: securing the insertion cord in a straight configuration prior to activating the wire tensioner; and securing the steering actuator position prior to activating the wire tensioner.

This could further reduce a potential risk of variance of the product, e.g. variance in tension of wires leading to different bending performance or non-straight configuration of the insertion cord.

A wire tensioner may comprise a linear actuator, rotary motors, and other options. Linear actuators are presently considered a relatively simple and efficient solution.

The linear actuators may comprise force sensors, which could enable feedback and reduce variance.

The pull force may be in the interval of 1-5 N, such as 2.5-3.5 N, which is presently considered to provide good results. For example, a pull force in this interval results in a moderate pre-tension of the steering wire significantly reducing the risk of slack even after extended shelf life, which may lead to some relaxation and creep of the components.

To further reduce any potential risk of variance the two force sensors and actuators may be coupled and configured to follow each other in matched motion. By matched motion is meant that the motion of the first and second wire grippers are linked but not necessarily synchronous.

The maximum difference in pull force between the two sensors during tensioning of the wire may be 1 N, which is presently considered to provide good results. This could be effected by continuous supervision of the pull force to ensure that the pull force does not exceed 1 N. If the pull force of one actuator exceeds the pull force of the other actuator by 1 N, the actuator with the highest pull force should slow down or stop until the other actuator catches up.

In an embodiment according to the second aspect, the endoscope comprises a wire directional guide arranged at the handle's interior. A wire directional guide could facilitate proper tensioning of the wire in that pull direction of the wire could be altered to have a specified direction.

In one embodiment according to the third aspect, the endoscope wire tensioning machine comprises: a wire tensioner and a wire fastener, wherein the wire tensioner comprises a first and second wire gripper, and the wire tensioner is adapted for matched motion of the first and second wire gripper.

Such a wire tensioning machine may automate production of endoscopes resulting in efficient endoscope production with low variance.

According to a variation of the foregoing embodiment, each wire tensioner comprises an actuator and a force sensor, and the force sensors are coupled to provide feedback to a matched motion controller for matched motion of the first and second wire gripper.

According to a variation of the foregoing embodiments and variations thereof, the endoscope wire tensioning machine further comprises a machine bed holding the wire tensioner and wire fastener, and a separate endoscope fixture adapted for holding a semi-assembled endoscope.

The “steering wire” may be referred to as “wire” throughout the description for simplicity.

### BRIEF DESCRIPTION OF THE DRAWINGS

The disclosure will now be made in greater detail based on non-limiting examples and with reference to the schematic drawings on which:

FIG. 1 shows a side view of an endoscope connectable to a monitor to form a visualization system,

FIG. 2 shows the interior of an endoscope handle,

FIG. 3 is a sketch of the outline of a wire in the handle of FIG. 2,

FIG. 4 shows the interior of a variant endoscope handle, schematically illustrating tensioning and fastening of the wire,

FIG. 5 is a perspective view of a wire tensioning machine according to the disclosure,

FIG. 6 is a side view of the wire tensioning machine of FIG. 5,

FIG. 7 is a close-up perspective view of the wire tensioning machine as depicted in FIG. 6,

FIG. 8 is a sectional view of the endoscope handle as mounted in the wire tensioning machine and depicted in FIG. 7,

FIG. 9 is a perspective view showing a detail of the wire routing in the wire tensioning machine,

FIG. 10 is a plan view schematically illustrating a wire gripping step in the wire tensioning method,

FIG. 11 is a plan view schematically illustrating a wire tensioning step in the wire tensioning method,

FIG. 12 is a sketch showing alternative routing of the wire during tensioning according to a first variant,

FIG. 13 is a sketch showing alternative routing of the wire during tensioning according to a second variant,

FIG. 14 is a sketch showing alternative routing of the wire during tensioning according to a third variant,

FIG. 15 is a flowchart of an example method of tensioning steering wires, and

FIG. 16 is a schematic depiction of a control system for the wire tensioning machine of FIG. 5.

### DETAILED DESCRIPTION

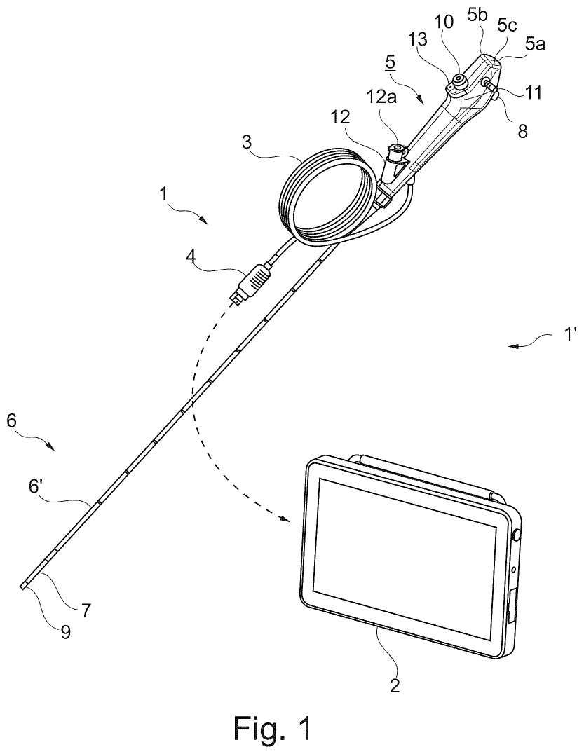

FIG. 1 illustrates a system 1′ comprising an endoscope 1 and a monitor 2. The endoscope 1 comprises an umbilical cord 3 and an electrical connector 4 for connecting to the monitor 2. The endoscope 1 is preferably single-use i.e. intended to be thrown away after use on one single patient, whereas the monitor 2 may be used multiple times with different single-use endoscopes. The endoscope 1 further comprises a handle 5 at a proximal end of the endoscope and an insertion cord 6 extending distally of the handle 5. The handle comprises a housing 5a that may be formed by attaching two shells 5b,5c to each other. The insertion cord 6 comprises an insertion tube 6′, a bending section 7, and a distal tip 9 with optical system. The bending section 7 may be controlled by a steering actuator 8 to deflect the distal end of the insertion cord 6. The optical system generally comprises a camera and an illumination emitter, such as light emitting diodes (LEDs) or optical fibers. As an alternative to arranging LEDs in the distal tip, the optical fibres may be provided for transporting light from a light source at the handle or outside the endoscope through the endoscope to the distal end thereof for illuminating an area of interest in front of the endoscope distal distal tip 9. The insertion cord 6 may comprise one or more channels, e.g. a working channel for advancing tools through the endoscope and out through an opening at the distal end of the endoscope, and a fluid pathway, such as a suction channel. The suction channel may be used for transporting fluids away from a position at the distal end of the endoscope, through an opening at the distal end of the endoscope. The same channel may be used as working channel and suction channel. The handle 5 often also comprises a suction valve button 10 comprising an external valve cap forming a push-button for activating suction through the suction channel to an external vacuum source (not shown) via a vacuum hose connector 11. The handle 5 may as shown also comprise a biopsy port 12 with a biopsy cap 12a for insertion of an external tool through the handle and the working channel to exit at the opening at the distal end of the endoscope. Further, the handle 5 may be provided with buttons for activation of electrical switches 13 controlling function of the image capture, such as taking of still images.

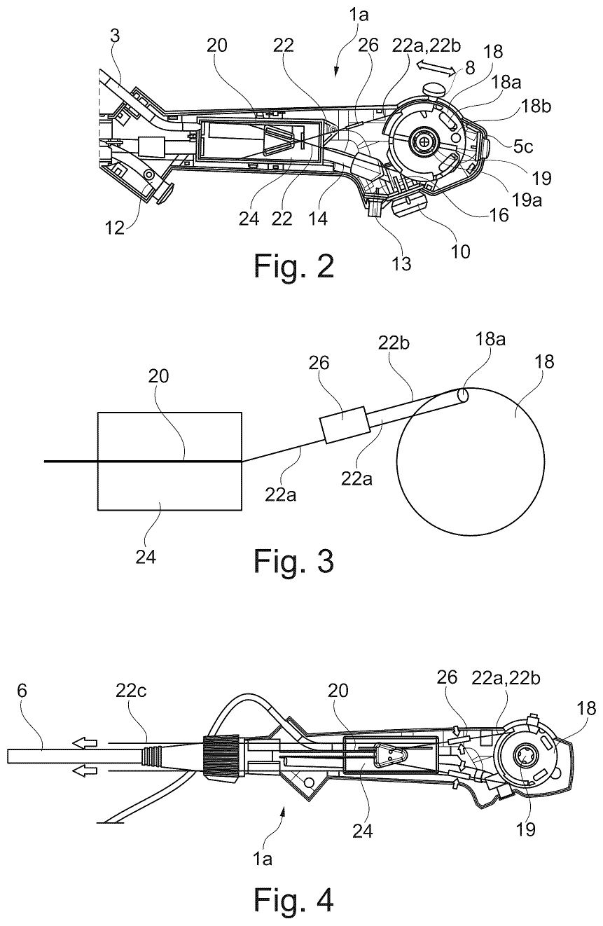

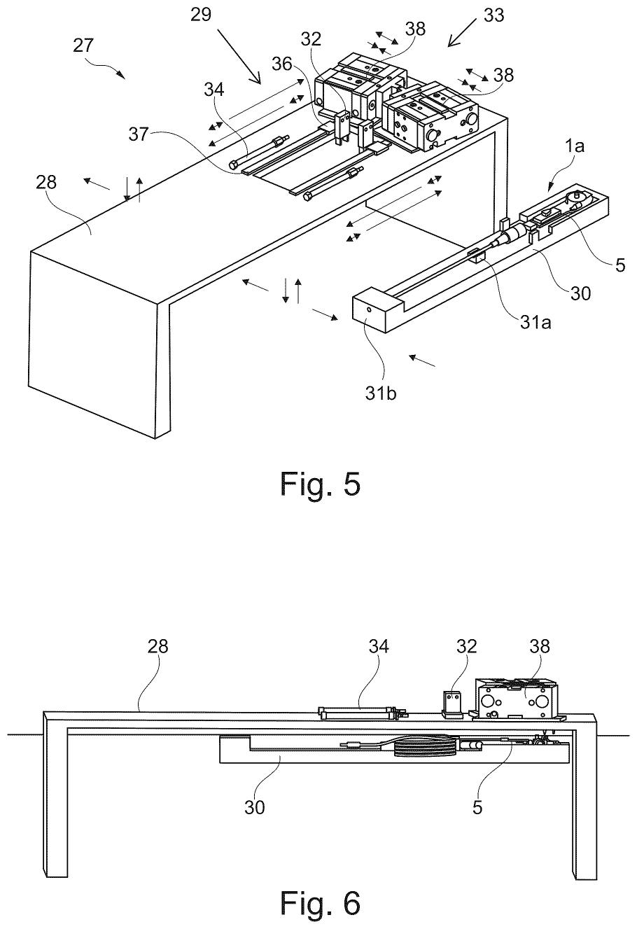

The interior of the handle 5 is illustrated in FIG. 2 showing an endoscope sub-assembly 1a. The wire layout is schematically illustrated in FIG. 3 showing only one wire for simplicity. As indicated, the handle 5 comprises two half shells 5b,5c of which one half shell (shown as 5c) can be used for fitting all handle components during assembly, whereas the other half shell, e.g. 5b, in this respect merely functions as a lid to close and form the handle. The illustrated suction valve button 10 activates a suction valve 16 to provide suction through a suction tube 14 connected to a suction channel running to the distal end of the endoscope 1 to remove fluids from a site inside a patient. The steering actuator 8 is connected to a roller 18 rotatably mounted on an axle 19, and a pair of steering wires 22 are connected to the roller 18. The axle 19 may be a cylindrical protrusion formed in one piece with the handle shell and comprising an opening 19a therethrough suitable to receive an alignment pin to align the handle shell in the wire tensioning machine, as described below with reference to FIGS. 5-8. The roller 18 may comprise an opening 18b perpendicular to its major surface and suitable to receive an alignment pin to align the roller in the wire tensioning machine, as described below with reference to FIGS. 5-8. Each wire 22 is part of a Bowden cable comprising also a wire pipe 20 covering each wire most of the way. The wire pipe 20 may be extending between, and fixedly attached to, an anchoring block 24 and a proximal end of the bending section, e.g. by means of adhesive or heat stacking. Pushing the steering actuator 8 in one direction or the other as indicated by the arrow effects pull of one of the two wires 22, which in the other end is connected to the bending section of the insertion cord, so the bending section will deflect. The wire pipes 20 terminate shortly after the anchoring block 24 in direction of the roller 18, leaving the steering wires 22 exposed towards the roller 18. In the example shown each steering wire 22 comprises a first steering wire portion 22a extending all the way from the bending section 7 to the roller 18, where the steering wire is connected at a connection point 18a, e.g. a hook, makes a U-turn back and continues back as a second steering wire portion 22b to a crimp 26 fixedly connecting the first and second steering wire portions 22a and 22b. The crimp 26 may be formed by a crimp shell, such as a F-75-10-M Ferrule distributed by Panduit.

In FIG. 3, the wire pipe 20 is fixedly connected to the anchoring block 24, whereas the steering wire 22 runs inside the wire pipe 20 and extends out in the direction of the roller. The steering wire comprises the first steering wire portion 22a, which connects to the roller 18 at connection point 18a. The steering wire then makes a U-turn and continues back as the second steering wire portion 22b and the first and second steering wire portions are fixedly connected by the crimp 26. The connection point 18a may be a hook, a hole through which the wire can be threaded, or any protrusion or recess configured to support the U-turn portion and secure the wire portion to the steering actuator 8 or the roller 18 of the steering actuator 8.

Details of a wire layout and tensioning are schematically illustrated in FIG. 4. Some details of the handle interior are slightly different from the illustration in FIG. 2. The anchoring block 24, for example, has a slightly different design but basically the same functionality, and the wire pipe 20 is again fixed to the anchoring block. The wire pipes 20 shown in FIG. 2 are forming large curves in the handle and the steering wires 22 are crossing after the anchoring block in a direction of the roller 18, whereas in FIG. 4 the wire pipes 20 are straight, and the steering wires 22 are not crossing. These different designs may be influenced by space considerations and illustrate that the method can be applied with different endoscopes having handles of different sizes and shapes. The endoscope sub-assembly 1a illustrated in FIG. 4 comprises steering wires extending out of the wire pipes 20 leaving the steering wires 22 exposed towards the roller 18. As explained above, the steering wire 22 comprises a first steering wire portion 22a extending to the roller 18, where the steering wire is connected, makes a U-turn back and continues back as a second steering wire portion 22b through a crimp shell and extending therefrom as a third steering wire portion 22c. As illustrated by arrows, the third steering wire portion 22c may be gripped and pulled to tension the steering wire 22 to a desired pre-tension and kept tensioned, and, as indicated by arrows, the crimp shell may subsequently be pressed against the first and second steering wire portions 22a, 22b running inside the crimp shell to permanently deform the crimp shell and forming the crimp 26 fixedly connecting the first and second steering wire portions 22a and 22b. The third steering wire portion 22c, which after the crimping is now in excess, may be cut at any suitable position, e.g. close to the crimp 26.

A wire tensioning machine 27 is in the following discussed with reference to FIGS. 5-9. The machine comprises a machine bed 28 forming a base structure or foundation for a wire tensioner 29 and a wire fastener 33. The endoscope sub-assembly 1a with the handle 5 may be secured directly to the machine bed or, as illustrated, indirectly via a separate fixture 30, which may comprise insertion cord holders 31a and 31b that maintain the insertion cord straight.

In the illustrated embodiment the wire tensioner 29 comprises a set of wire grippers 32 to grip the steering wire in the endoscope sub-assembly. The wire grippers 32 are mounted on a slide 36 to be movable along rails 37 by means of actuators 34, e.g. pneumatic cylinders or linear actuators comprising worm gears rotated by motors, to thereby tension the steering wire 22. As an example, the wire gripper may be a parallel gripper DHPS from the company Festo.

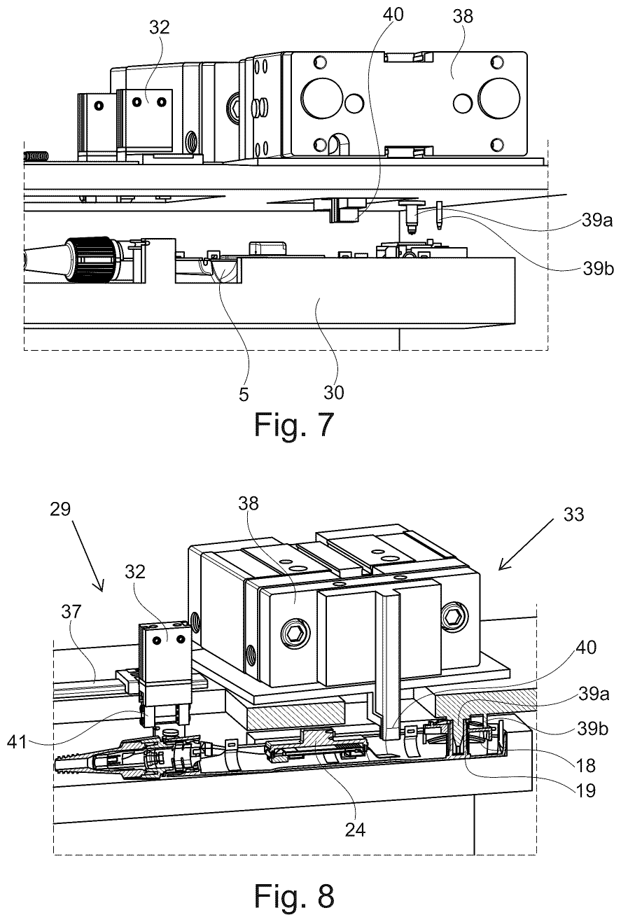

The machine further comprises a wire fastener 33, which in the illustrated embodiment comprises a set of crimpers 38 with crimper jaws 40 (FIG. 7) for deforming the crimp shells to form crimps, thereby fastening the wire. Preferably the crimper jaws 40 undergo a linear movement towards each other when they close.

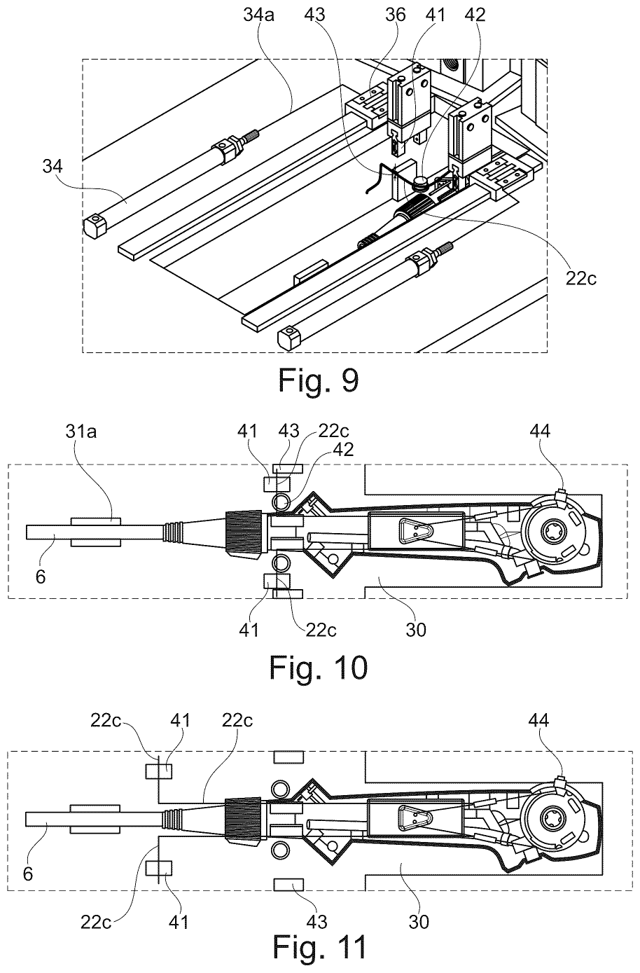

Movement and hence functioning of the machinery is illustrated by arrows in FIG. 5 and will be explained in more details with reference also to the previous figures. The fixture 30 with the endoscope sub-assembly 1a may be introduced from one side, e.g. on a conveyor, to a position underneath the machine bed 28, as illustrated in the side view of FIG. 6, and lifted into position in the wire tensioning machine. FIG. 7, being a close-up detail of FIG. 6, shows alignment pins 39a and 39b which may be provided to ensure proper positioning of the endoscope handle 5 relative to the machine bed 28, facilitating automation and lowering variance of endoscopes produced. The alignment pins 39a and 39b may engage corresponding openings 18b,19a of the endoscope handle, such as at the axle 19 and in the roller 18, as illustrated in FIG. 8, showing the fixture in position for tensioning. In a next step, gripper jaws 41 of the wire grippers 32 grip third steering wire portion 22c of the steering wire 22 as illustrated in FIG. 9, which for reasons of simplicity shows only one third steering wire portion 22c. The fixture comprises a wire presenter 42 and a wire holder 43. The wire presenter 42 and wire holder 43 releasably present and hold the third steering wire portion 22c in a position for the gripper jaws 41 of the wire gripper 32 to grip the wire. This is also schematically illustrated in FIG. 10 showing the fixture with the endoscope sub-assembly in the moment where the third steering wire portion 22c is gripped. Then the wire grippers 32 will travel along rails 37 activated by pneumatic cylinders 34 connected by linkage 34a to the slide 36 as schematically illustrated in FIG. 9 and with reference also to FIG. 11 to thereby pull and hence tension the steering wire. The gripper jaws may be comprised of the jaws of the parallel gripper DHPS and attachments thereto extending the reach so that different attachments/extenders can be used for different endoscopes without modifying the machine or requiring different parallel gripper DHPS.

As can be seen in FIG. 10, the fixture 30 may comprise an actuator fixation 44 to hold the steering actuator 8 in a neutral position. Further the fixture comprises the insertion cord holder 31a,31b holding the insertion cord 6 in a straight configuration.

The gripper jaws 41 may grip the steering wire at right angles to the direction of travel, which may lower the risk of slippage.

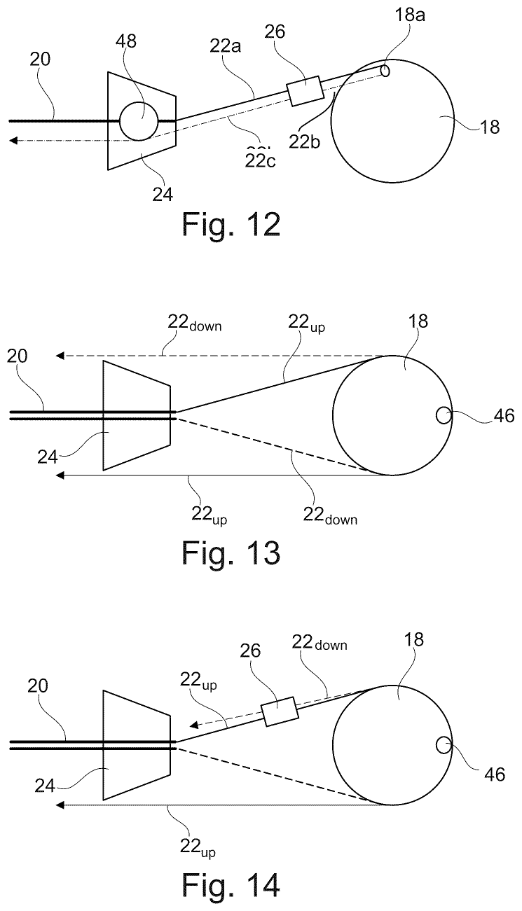

FIGS. 12 to 14 schematically illustrate different variants with alternative wire routing and fixation, which will be explained in more detail below. FIG. 12, which, for the sake of clarity, illustrates only one wire, illustrates the provision of a wire direction guide 48. Again, the wire pipe 20 is fixedly attached to the anchoring block 24. The first steering wire portion 22a extends out of the wire pipe 20, attaches to the connection point 18a of the roller 18, loops back as second steering wire portion 22b, illustrated as a dash dot line, to the crimp 26 and continues as third steering wire portion 22c. The third steering wire portion 22c engages and changes direction around the wire direction guide 48. Hereby is ensured that the pull direction of the first steering wire portion 22a and second steering wire portions 22b are substantially parallel, which lowers the risk of incorrect tensioning of the steering wire.

FIGS. 13 and 14 illustrate alternative wire routings and fixation. In FIG. 13 the wire pipes 20 are fixedly connected to the anchoring block 24. An upper steering wire for the up-direction, 22ᵤₚ, shown in solid line, extends from the wire pipe 20 to the roller 18, follows the roller 18 and returns as indicated, and with pull direction as indicated by the arrow. Similarly, a lower steering wire for the down-direction, 22_{down}, shown in dashed line, extends from the wire wipe to the roller 18, follows the roller 18 and returns as indicated, with the pull direction indicated by the arrow. Instead of the steering wire forming a loop and being fixed to itself as discussed above, the steering wire may alternatively be fixedly connected directly to the roller 18 at a wire anchorage 46, e.g. by a screw, a rivet, glue or by heat stacking, whereby the previously described crimp is avoided. Tension of the steering wires are kept until the wires are fixedly connected to the roller 18. The steering wires may be moved relative to the roller and thereby positioned on the roller to achieve the desired shape of the insertion cord and roller position before the steering wires are fixated, which may be an advantage. Each wire may be individually fixedly connected to the roller 18, but in the simplest variant the wire anchorage 46 fixedly connects steering wires for both directions to the roller at the same time, so this solution uses a minimum number of parts and hence the method and resulting endoscope is particularly simple.

In the variant of FIG. 14 the two steering wires are routed essentially as explained with reference to FIG. 13, but connecting the two wires to each other, e.g. by the crimp 26, thus forming a wire loop around the roller 18. The wire loop may slip on the roller 18 thereby allowing adjustment of the position of the steering actuator. Subsequently this combined wire set may be fixed to the roller 18 at the anchorage point 46.

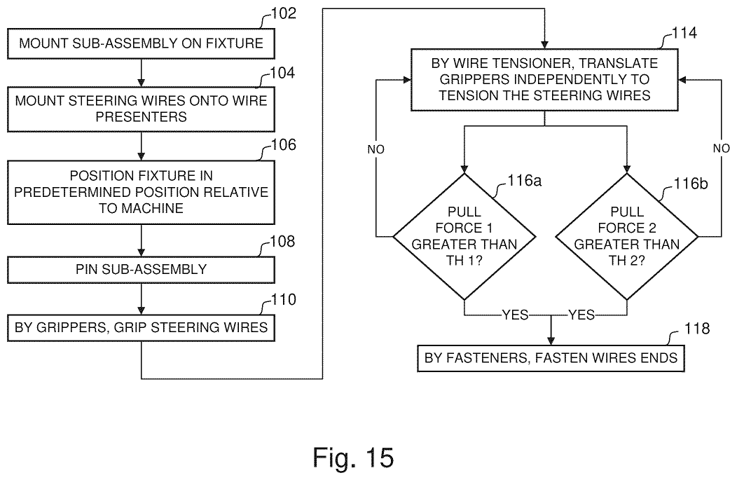

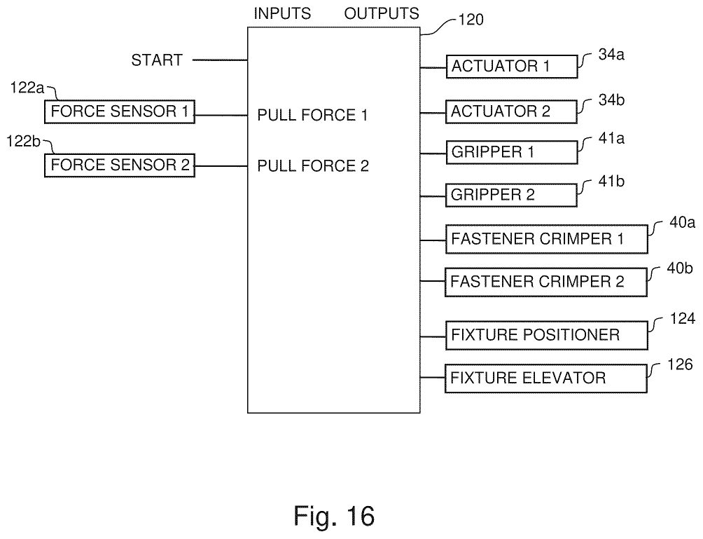

FIG. 15 shows a flowchart depicting an example, according to the first aspect, of the method for tensioning steering wires of an endoscope with a tensioning system including a tensioning machine. The tensioning machine may comprise a control system, such as the one schematically depicted in FIG. 16. In the present example, an endoscope sub-assembly is mounted on a fixture, at 102. The sub-assembly comprises one half of the housing of the handle, e.g. a half-shell of the housing. In the half-shell is the steering actuator including a roller to which ends of the steering wires are or will be affixed. As described with reference to FIG. 2, the steering wires translate inside wire pipes fixedly attached, at their proximal ends, to the anchoring block. The first steering wire portions 22a extend proximally of the proximal ends of the wire pipes towards the roller, where they are connected, at least slidingly, at a connection point 18a. If a crimp shell is used, the first steering wire portions 22a are threaded through the crimp shell before the connection point 18a. The steering wires are then bent back toward to the bending section by the operator and are now referred to as the second steering wire portions 22b. If a crimp shell is used, the second steering wire portions 22b are threaded through the crimp shell in parallel with the first steering wire portions 22a, as shown in FIG. 12. The tails extending distally from the crimp shell are now referred to as the third steering wire portions 22c. The third steering wire portions 22c are placed on the wire presenters by the operator.

Prior to positioning the steering wires onto the half-shell, other components of the endoscope, particularly components that fit between the steering wires and the half-shell, are placed onto the half-shell. These may comprise the working channel tube, an irrigation tube, etc. The insertion cord is already attached to the half-shell, as shown in FIGS. 10 and 11. The fixture may comprise holders 31a,31b for the insertion cord and the half-shell. The insertion cord holder 31a may comprise a U-shape having a slot so that the insertion cord can be slipped into the slot. The insertion cord holder 31b may comprise a block with a hole through which the distal tip is inserted. More than one insertion cord holder 31a may be used, particularly if the insertion cord is long. The purpose of the holders is to keep the insertion cord straight during the tensioning process. The half shell holder may comprise a cavity shaped like the half-shell to make the process of securing the sub-assembly in the fixture simpler and faster. An actuator fixation/fixture can be used to hold the sub-assembly in place. The fixture may comprise a spring-loaded detent that presses onto the steering actuator to hold it in place. The detent may comprise a cavity shaped to receive the steering actuator.

The steering wires of the sub-assembly, i.e. the third steering wire portions 22c, are mounted onto wire presenters, at 104, by an operator. This is preferably done after the sub-assembly is mounted but can also be done as an initial mounting step. Wire presenters 42 and wire holders 43 are shown in FIG. 10. The wire presenters may comprise protrusions that extend from the fixture on both sides of the half-shell so that the third steering wire portions 22c can be bent away from a central axis of the endoscope. To prevent kinking, the wire presenters comprise curved surfaces. As shown, the wire presenters are cylindrical pillars. The wire presenters may also function to hold the half-shell in place. For that purpose the fixture is designed to tension a particular endoscope type/size/shape while the overall machine is designed to tension different endoscope types/sizes/shapes. The wire holders may comprise blocks with slits. The blocks may be made of a synthetic resilient material and the slits sized to removably receive the steering wires and to hold them. The wire holders are positioned laterally from the wire presenters so that a portion of the third steering wire portions 22c extends between them, the portion being sufficiently long to permit a wire gripper to grip it.

Having secured the sub-assembly to the fixture, at 106 the fixture is positioned in a predetermined position relative to the machine. To enhance safety and repeatability, the fixture may be mounted onto a moving bed that is configured to move under the bed of the machine and up. Actuators may be used to perform the positioning so that the operator does not have to reach below the tensioning machine. The actuators may be pneumatic actuators. In FIG. 16, fixture positioners 124 and 126 are depicted. These may be electrical switches activated by a controller 120 to engage/disengage the fixture actuators. In one example, the fixture positioners actuate pneumatic valves that cause the pneumatic actuators to extend laterally, by fixture positioner 124, and vertically, by fixture positioner 126. Electrical linear actuators may be used instead. Any type of actuator that can translate the fixture laterally and/or vertically may be used. Of course, such positioning may also be performed by the operator pushing the fixture under the machine bed and using a lever to raise the fixture.

The final position of the sub-assembly may be refined by pinning the sub-assembly to the machine, at 108. To this end the machine has pins that fit into apertures of the axle and/or roller, shown in FIG. 8. The ends of the pins are tapered to “find” the apertures. As the sub-assembly is raised by the fixture, the tapered portion and or the cylindrical surfaces of the pins contact the cylindrical surfaces of the apertures and may slightly move the sub-assembly during this process. Of course, other means can be used to pin the sub-assembly, such as downward protrusions that engage inner surfaces of the half-shell, etc. These downward protrusion pins do not have to be cylindrical and may comprise rectangular shapes, for example, with tapered surfaces parallel to the wall of the half-shell.

After the sub-assembly is secured in its final position, the tensioning process can begin, at 110, by activating the wire grippers to grip the third steering wire portions 22c located between the wire holders and the wire presenters. The wire grippers may comprise pneumatic cylinders activated by pneumatic valves engaged by electrical switches and solenoids, denoted as gripper 1 41a and gripper 2 41b in FIG. 16.

At 114, the wire tensioner translates the wire grippers independently to tension the steering wires. The wire tensioner may comprise pneumatic cylinders activated by pneumatic valves (e.g. actuators) engaged by electrical switches and solenoids, denoted as actuator 1 34a and actuator 2 34b in FIG. 16. In one example, the tensioner translates a predetermined distance. In another example, the tensioner translates each gripper to a predetermined pressure in the pneumatic cylinder. In another example, the tensioner translates each gripper to a predetermined pull force by sensing the pull force with force sensors 1 and 2, 122a, 122b, shown in FIG. 16. When the pull force determined by the controller 120 exceeds a respective threshold, at 116a, 116b, the controller 120 causes the tensioner to stop translating the respective wire gripper by disengaging the respective output.

The controller 120 may cause the tensioner to engage the wire grippers and actuators concurrently or sequentially. It is preferred if both wire grippers translate at about the same time so that bending tension on the insertion cord is neutralized by pulls on both sides. Due to natural machine delays one wire gripper may start translating before the other, but preferably they translate concurrently during most of their travel and at substantially the same speed so that there is a minimal tension difference between the wires during tensioning. For example, the wire grippers may translate concurrently for at least 75% of the translation distance. The translation distances may be different, in which case at least 75% of a distance translated by the first wire gripper and at least 75% of a distance translated by the second wire gripper are translated concurrently. The minimal tension difference may be 10% of a desired final pull force or tension. The desired tension may be the tension at or above the pull force threshold and the steering wire may be said to be in a tensioned state when such tension threshold is reached.

Once the steering wires are tensioned, the fasteners can fasten the steering wires to maintain the tension, at 118. In one example, fastening comprises crimping the crimp shells. This is done by the fastener crimpers, which may comprise pneumatic cylinders activated by pneumatic valves (e.g. actuators) engaged by electrical switches and solenoids, denoted as fastener crimper 1, 2 40a,40b in FIG. 16. In another example, the wires are secured/fastened to the roller, as described above, and the fastener crimper 1, 2 activate respective fastening means.

In another example according to the first aspect, the sub-assembly is placed by the operator on the tensioning machine without a fixture and the bed of the tensioning machine comprises the wire presenters 42 and the wire holders 43. The tensioner works in the same way as in the previous example. In this example the tensioning machine is simpler, thus less expensive, which may be appropriate for tensioning endoscopes produced in low volumes.

The tensioning machine may comprise a control system including a controller 120. The controller 120 may comprise a programmable logic controller (PLC) suitably programmed to receive input signals and generate output signals, as is known in the art. PLCs are inexpensive and commonly available. The controller may also comprise a computer with software and connected to an input/output (I/O) module. The controller may also comprise a dedicated integrated circuit, such as a field-programmable-field-array (FPGA). Any other type of control device configured to received inputs and generate outputs may be used. In FIG. 16 the inputs and outputs are depicted schematically. The inputs include a start signal, a force sensor 1 signal, and a force sensor 2 signal. The start signal may be generated by a start button which the operator actuates to initiate tensioning. If step 106 is conducted by an operator manually, the start signal would initiate operation of the wire tensioner, at 110. If step 106 is performed automatically, the start signal would initiate movement of the fixture, at 106. The function of the force sensors was explained above. The force sensors may be strain gages, MEMS, etc.

Additional inputs may comprise position detecting sensors (optical or contact sensors), safety switches and the like. The outputs include the actuators 1,2 34a,34b, the gripper 1, 2 41a,41b, the fastener crimpers 1, 2 40a,40b, and the the fixture positioners 124, 126. The functions of the outputs were explained above.

Although exemplified with a two-way endoscope bendable in two directions the principles of the invention could also be advantageous in relation to four-way endoscopes bendable in four directions. In such case, the tensioning machine would additionally include two additional wire presenters 42, wire holders 43, and wire grippers 32.

The following items are further variations and examples of the embodiments described with reference to the figures.

1. Method for tensioning steering wires of an endoscope comprising: providing a semi-assembled endoscope comprising a handle and an insertion cord extending in a distal direction from the handle, the insertion cord having a distal bending section connected to a first steering wire and a second steering wire, each steering wire having a first wire end being connected at the bending section at a distal end thereof and arranged in a respective wire pipe extending from a proximal end of the bending section to the handle, each steering wire having a proximal end extending further out of the wire pipe; providing a wire tensioner comprising a first and second wire gripper; providing a wire fastener; securing the semi-assembled endoscope relative to the wire tensioner and wire fastener; arranging each wire gripper to grip a respective one of the steering wires; activating the wire tensioner to tension the steering wires; and activating the wire fastener to fixate the steering wires in the tensioned state.

2. Method according to item 1, wherein some tensioning of one wire occurs concurrently with at least some tensioning being performed on the other wire.

3. Method according to item 1 or 2, wherein fixation of the steering wires is performed by crimping.

4. Method according to any one of the items 1-3, comprising: providing a separate endoscope fixture; securing the semi-assembled endoscope to the fixture; and matching the semi-assembled endoscope on the fixture to the wire tensioner and wire fastener.

5. Method according to any one of the items above, comprising: securing the insertion cord in a straight configuration prior to activating the wire tensioner; and securing the steering actuator position prior to activating the wire tensioner.

6. Method according to any one of the items above, wherein each wire tensioner comprises a linear actuator.

7. Method according to item 6, wherein the linear actuators comprise force sensors.

8. Method according to item 6 or 7, wherein the pull force is in the interval of 1-5 N, such as 2.5-3.5 N.

9. Method according to item 7 or 8, wherein the two force sensors and actuators are coupled and configured to follow each other.

10. Method according to item 9, wherein the maximum difference in pull force between the two sensors during tensioning of the wire is 1 N.

11. Endoscope manufactured using the method according to any one of the items above.

12. Endoscope according to item 11, wherein the endoscope comprises a wire directional guide arranged at the handle interior.

13. Endoscope wire tensioning machine comprising: a wire tensioner and a wire fastener, the wire tensioner comprising a first and second wire gripper, wherein the wire tensioner is adapted for matched motion of the first and second wire gripper.

14. Endoscope wire tensioning machine according to item 13, wherein each wire tensioner comprises an actuator and a force sensor, and the force sensors are coupled to provide feedback to a control for matched motion of the first and second wire gripper.

15. Endoscope wire tensioning machine according to item 13 or 14, further comprising a machine bed holding the wire tensioner and wire fastener, and a separate endoscope fixture adapted for holding a semi-assembled endoscope.

### LIST OF REFERENCE SIGNS

1 Endoscope
1a Endoscope sub-assembly
2 display unit
3 umbilical cord
4 electrical connector
5 handle
6 insertion cord
7 bending section
8 steering actuator
9 distal tip
10 suction valve button
11 vacuum hose connector
12 biopsy connector
12a biopsy cap
13 electrical switch
14 suction tube
16 suction valve
18 roller
18a connection point
19 axle
20 wire pipe
22 steering wire
22a first steering wire portion
22b second steering wire portion
22c third steering wire portion
22 down steering wire, down
22 up steering wire, up
24 anchoring block
26 crimp
28 machine bed
29 wire tensioner
30 fixture
31a insertion cord holder
31b insertion cord holder
32 wire gripper
33 wire fastener
34 actuator
34a linkage
36 slide
37 rail
38 wire crimper
39a alignment pin
39b alignment pin
40 crimper jaws
41 gripper jaws
42 wire presenter
43 wire holder
44 actuator fixation
46 wire anchorage
48 wire direction guide

1. A method for tensioning steering wires of an endoscope, the method comprising:
assembling a semi-assembled endoscope, the semi-assembled endoscope comprising a half-shell of a handle, a steering actuator, and an insertion cord extending in a distal direction from the half-shell, the insertion cord including an insertion tube and a bending section, the bending section connected to a first steering wire and a second steering wire, each of the first steering wire and the second steering wire comprising a first wire portion, a third wire portion, and a second wire portion between the first wire portion and the third wire portion, each first wire portion being connected to the bending section and arranged in a respective wire pipe extending through the insertion tube to the handle, and each second wire portion extending proximally out of the respective wire pipe;
positioning the sub-assembly in a predetermined position in a wire tensioning machine, the wire tensioning machine comprising a controller, a first wire tensioner including a first wire gripper, a second wire tensioner including a second wire gripper, a first wire fastener, and a second wire fastener;
the controller causing:
the first wire gripper to grip the third wire portion of the first steering wire;
the second wire gripper to grip the third wire portion of the second steering wire;
the first wire tensioner to translate the first wire gripper distally to tension the first steering wire to a first tensioned state;
while translating the first wire gripper, the second wire tensioner to translate the second wire gripper distally to tension the second steering wire to a second tensioned state;
the first wire fastener to fixate the first steering wire in the first tensioned state; and
the second wire fastener to fixate the second steering wire in the second tensioned state.

2. The method of claim 1, wherein at least 75% of a distance translated by the first wire gripper and at least 75% of a distance translated by the second wire gripper are translated concurrently.

3. The method of claim 1, wherein the wire tensioning machine comprises force sensors to sense a first pull force in the first steering wire and a second pull force in the second steering wire, and wherein the first pull force and the second pull force do not differ by more than 25% while the the first wire gripper and the second wire gripper are translated.

4. The method of claim 1, wherein the wire tensioning machine comprises force sensors to sense a first pull force in the first steering wire and a second pull force in the second steering wire, and wherein the first pull force and the second pull force do not differ by more than 1 N.

5. The method of claim 1, wherein the first wire fastener comprises a first crimper configured to crimp a first crimp shell to fixate the second wire portion and the third wire portion of the first steering wire to each other, and wherein the second wire fastener comprises a second crimper configured to crimp a second crimp shell to fixate the second wire portion and the third wire portion of the second steering wire to each other.

6. The method of claim 1, wherein the first and second wire fasteners fixate the first steering wire and the second steering wire, respectively, by crimping.

7. The method of claim 1, wherein positioning the sub-assembly in the predetermined position in the wire tensioning machine comprises:
securing the semi-assembled endoscope onto a fixture; and
moving the fixture to place the sub-assembly in the predetermined position.

8. The method of claim 7, wherein securing the semi-assembled endoscope onto the fixture comprises:
securing the insertion cord in a straight configuration to the fixture; and
securing the steering actuator position.

9. The method of claim 7, wherein securing the semi-assembled endoscope onto the fixture comprises:
securing the insertion cord in a straight configuration to the fixture;
securing the steering actuator position;
securing the third portion of the first steering wire to a first wire holder of the fixture; and
securing the third portion of the second steering wire to a second wire holder of the fixture.

10. The method of claim 1, wherein positioning the sub-assembly in the predetermined position in the wire tensioning machine comprises:
positioning a fixture in a non-overlapping position relative to a bed of the wire tensioning machine;
while in the non-overlapping position:
securing the insertion cord in a straight configuration to the fixture;
securing the steering actuator position on the fixture;
securing the third portion of the first steering wire to a first wire holder of the fixture; and
securing the third portion of the second steering wire to a second wire holder of the fixture; and
translating the fixture until it overlaps with the bed with the first wire gripper and the second wire gripper overlapping, at least in part, the third wire portion of the first steering wire and of the second steering wire, respectively.

11. The method of claim 1, wherein positioning the sub-assembly in the predetermined position in the wire tensioning machine comprises:
positioning a fixture in a non-overlapping position relative to a bed of the wire tensioning machine;
securing the insertion cord in a straight configuration to the fixture;
securing the steering actuator position on the fixture;
securing the third portion of the first steering wire to a first wire holder of the fixture;
securing the third portion of the second steering wire to a second wire holder of the fixture;
translating the fixture until it overlaps with the bed with the first wire gripper and the second wire gripper overlapping, at least in part, the third wire portion of the first steering wire and of the second steering wire, respectively; and
raising the fixture to pin the sub-assembly in place.

12. The method of claim 1, wherein positioning the sub-assembly in the predetermined position in the wire tensioning machine comprises:
positioning a fixture in a non-overlapping position relative to a bed of the wire tensioning machine;
securing the insertion cord in a straight configuration to the fixture;
securing the steering actuator position on the fixture;
securing the third portion of the first steering wire to a first wire holder of the fixture;
securing the third portion of the second steering wire to a second wire holder of the fixture;
translating the fixture until it overlaps with the bed with the first wire gripper and the second wire gripper overlapping, at least in part, the third wire portion of the first steering wire and of the second steering wire, respectively; and
raising the fixture to pin the sub-assembly in place,
wherein said translating and raising is caused by the controller.

13. The method of claim 1, wherein each wire tensioner comprises a linear actuator.

14. The method of claim 13, wherein the linear actuators comprise force sensors configured to sense pull forces.

15. The method of claim 14, wherein the pull forces are between 1 N and 5 N.

16. The method of claim 14, wherein the pull forces are between 2.5 N and 3.5 N.

17. The method of claim 14, wherein a maximum difference in pull force between the two sensors during tensioning of the steering wires is 1 N.

18. The method of claim 13, wherein the force sensors and actuators are coupled and configured to follow each other.
